(19) United States
(12) Patent Application Publication
(10) Pub. No.: US 2026/0367422 A1
(43) Pub. Date: Nov. 19, 2026

(54) Augmented Monofocal Ophthalmic Lens with Micro-Wavefront Perturbation

(71) Applicants: Aaren Scientific Inc., Rancho Cucamonga, CA (US); HENAN SIMEDICE BIOPHARMACEUTICAL CO., LTD., Zhenzhou, Henan (CN); HENAN UNIVERSE INTRAOCULAR LENS RESEARCH AND MANUFACTURE CO., LTD., Shenzhou, Henan (CN)

(72) Inventor: Yueai LIU, Rancho Cucamonga, CA (US)

(73) Assignee: Aaren Scientific Inc., Rancho Cucamonga, CA (US)

(21) Appl. No.: 19/133,847

(22) PCT Filed: Dec. 1, 2023
(86) PCT No.: PCT/US2023/082074

§ 371 Date: May 29, 2025

Related U.S. Application Data
(60) Provisional application No. 63/385,800, filed on Dec. 2, 2022.

(51) Int. Cl. G02C 7/02; A61F 2/16; G02C 7/04

(52) U.S. Cl. CPC G02C 7/028; A61F 2/164; G02C 7/04

(57) ABSTRACT

Disclosed is a monofocal ophthalmic lens, the performance of which is augmented for presbyopia correction, having a wavefront that is modified with a perturbation at its central paraxial region. The perturbation may be realized through surface relieving on either its anterior or posterior surfaces or through refractive index modification of the lens material. The perturbation is in the form of an even function such as but not limited to a cosine, Fourier harmonics, Zernike polynomials, or Taylor series with respect to the radius of the wavefront aperture.

### CROSS-REFERENCE TO RELATED APPLICATIONS

This application claims priority from U.S. Provisional Patent Application No. 63/385,800 filed on Dec. 2, 2022. The entire disclosure of the prior application is considered to be part of the disclosure of the accompanying application and is hereby incorporated by reference.

### BACKGROUND OF THE INVENTION

### 1. Field of the Invention

The present disclosure generally relates to ophthalmic lenses and, more specifically, to monofocal ophthalmic lenses having a wavefront perturbation on their central paraxial region.

### 2. Description of the Related Art

A common form of eye disease among the senior population is cataracts. A cataract is a cloudy area in the eye's lens that leads to decreased vision. Cataracts often develop slowly and can affect one or both eyes, often leading to difficulty performing daily tasks such as driving, reading, or recognizing faces. If left untreated, cataracts may lead to blindness. A common form of treatment is to remove the natural lens, called the crystalline lens, and replace it with a prosthetic lens called an intraocular lens (IOL). An IOL can be designed to provide an excellent visual acuity for a single focal point or for several focal points. The former is referred to as a monofocal IOL and generally uses an aspheric surface. In contrast, the latter is referred to as a multifocal IOL and uses a diffractive surface.

A multifocal IOL, having more than one focal point, is often preferred over a monofocal IOL as such lenses generally eliminate the need for glasses. However, multifocal lenses are more expensive than monofocal lenses because of their diffractive design, and patients using multifocal lenses are many times less satisfied with their vision experience than patients using monofocal lenses. As a result, many IOL monofocal designs seek to improve the depth of focus at the lens's single focal point by modifying its aspherical surface.

One example is United States publication 20200121448 to Myoung-Taek Choi et al. titled “Extended depth of focus intraocular lens” which discloses an IOL that comprises an optic zone and a modulated surface profile formed in the optic zone and configured to focus incident light at a plurality of focal points, wherein the modulated surface profile is incorporated with a base surface profile of the optic zone. The modulated surface may have a profile that is sinusoidal, triangular, or some form thereof, the purpose of which is to extend the depth of focus. Another example is U.S. patent Ser. No. 11/083,566 to Xin Hong et al. titled “Ophthalmic lens having an extended depth of focus.” Here, Hong discloses an ophthalmic lens that includes an optic having an anterior surface, a posterior surface, and an optical axis. At least one of the anterior surface and the posterior surface includes a first zone extending from the optical axis to a first radial boundary and a second zone extending from the first radial boundary to the edge of the optic. The first zone includes an inner region and an outer region separated by a phase shift feature, the phase shift comprising a ridge extending outwardly from the inner region and the outer region. The optical combination of the inner region, phase shift feature, and outer region of the first zone and the second zone extends the depth of focus of the lens.

Although the above approaches towards increasing the depth of focus of a monofocal lens are acceptable, the state of the art is open to other approaches that may lend themselves to extending the depth of focus while also simplifying the manufacturing of the lens, resulting in lower costs for wearing such a lens.

### BRIEF SUMMARY OF THE INVENTION

Disclosed herein is an ophthalmic lens such as spectacle glasses, contacts, or intraocular lenses having an optic that is generally monofocal but having a zone about the central paraxial region where a wavefront perturbation has been added. The wavefront perturbation could be realized with surface relieving on either the anterior and posterior surfaces of the lens or with refractive index modification of the lens material at some depth inside of the optic. The object of the wavefront perturbation is to extend the depth of focus that may correct the presbyopia of the wearer under photopic conditions at all distances. Under mesopic vision, the lens becomes monofocal-like.

For some background, FIG. 2 shows monofocal ophthalmic lens 10, the wavefront emerging from the lens is largely a sphere that converges to a single focal point, shown as 14 in FIG. 2. Lens 10 through focus Modulation Transfer Function (MTF) is shown as 16. Light converging at focal point 14 may be said to be emmetropia vision. It is desirable for a presbyopia or a pseudophakia patient who wears lens 10 to have a monofocal through focus MTF 16 that is wider, representing a greater depth of focus extending from the emmetropia focus toward the myopia direction.

FIG. 3 shows augmented monofocal ophthalmic lens 20 having a central paraxial region 26 of the present disclosure. Paraxial region 26 contains a wavefront perturbation that diffracts the wavefront emerging from lens 20 and splits it into numerous sub-wavefronts, allowing the incoming light energy to be distributed to a depth of focus from focal point 14 in the myopia direction. As a result, the width of through focus MTF 16 is increased, thereby its depth of focus. Such a wavefront-perturbed ophthalmic lens would let a presbyopia or pseudophakia wearer gain a pseudo accommodation function and see clearly within the depth of focus of the lens under photopic conditions.

What is disclosed herein is a monofocal ophthalmic lens 20 having perturbation within paraxial region 22. The perturbation is a continuous even function of the radius of paraxial region 22 and is rotationally symmetrical about the optical axis of lens 20.

Other features and advantages of various embodiments of the present invention will be apparent to one skilled in the art from the following description.

### BRIEF DESCRIPTION OF DRAWINGS

The present invention will become more fully understood from the detailed description and accompanying drawings. Other systems, methods, features, and advantages of the invention will be or will become apparent to one with skill in the art upon examination of the following figures and detailed description. It is intended that all such additional systems, methods, features, and advantages be included within this description, be within the scope of the invention, and be protected by the accompanying claims. Component parts shown in the drawings are not necessarily to scale and may be exaggerated to better illustrate the important features of the invention. Dimensions disclosed or shown are exemplary only. In the drawings, like reference numerals may designate like parts throughout the different views, wherein.

FIG. 1 shows the augmented monofocal ophthalmic lens of the present disclosure having a central paraxial region containing a micro wavefront perturbation;

FIG. 2 shows how a monofocal ophthalmic lens of the prior art functions;

FIG. 3 shows how the augmented monofocal ophthalmic lens of the present disclosure functions;

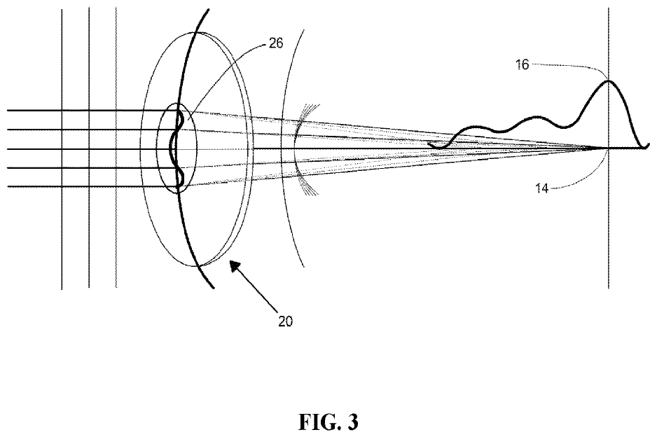

FIG. 4 shows the surface profile of a first embodiment where the perturbation is expressed as a cosine series;

FIG. 5 shows the through focus MTF of a first embodiment where the perturbation is expressed as a cosine series.

FIG. 6 shows the surface profile of a second embodiment where the perturbation is expressed as a cosine series;

FIG. 7 shows the through focus MTF of a second embodiment where the perturbation is expressed as a cosine series.

FIG. 8 shows the surface profile of a third embodiment where the perturbation is expressed as a Fourier harmonic series;

FIG. 9 shows the through focus MTF of a third embodiment where the perturbation is expressed as a Fourier harmonic series.

FIG. 10 shows the surface profile of a fourth embodiment where the perturbation is expressed as a Fourier harmonic series;

FIG. 11 shows the through focus MTF of a fourth embodiment where the perturbation is expressed as a Fourier harmonic series.

FIG. 12 shows the surface profile of a fifth embodiment where the perturbation is expressed as a Fourier harmonic series;

FIG. 13 shows the through focus MTF of a fifth embodiment where the perturbation is expressed as a Fourier harmonic series.

### DETAILED DESCRIPTION OF THE INVENTION

The exemplary embodiments relate to ophthalmic devices such as spectacle glasses, IOLs, and contact lenses. The following description is presented to enable one of ordinary skill in the art to make and use the invention and is provided in the context of a patent application and its requirements. Modifications to the exemplary embodiments and the generic principles and features described herein will readily be apparent. The exemplary embodiments are mainly described in terms of particular methods and systems provided in particular implementations. However, the methods and systems will operate effectively in other implementations. For example, the method and system are described primarily in terms of IOLs. However, the method and system may be used with contact lenses and spectacle glasses.

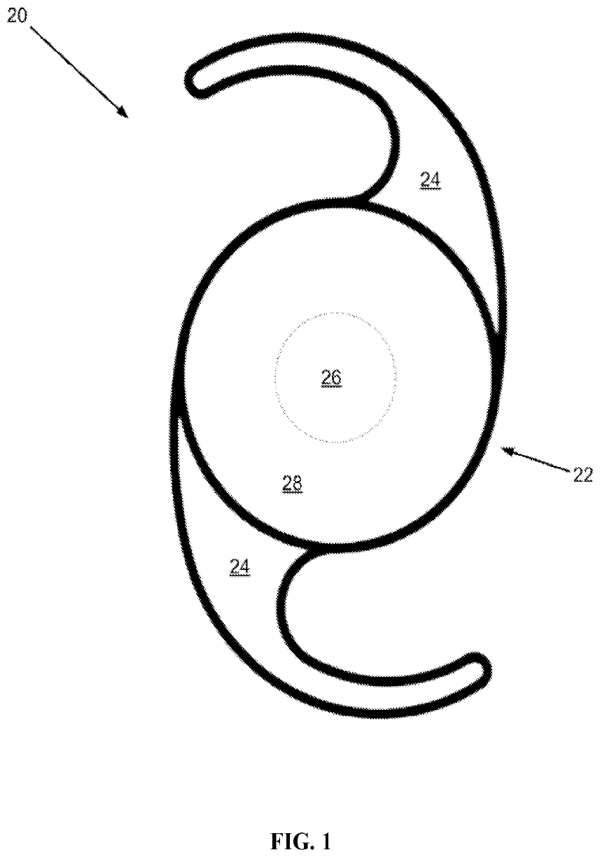

Disclosed herein is an augmented monofocal ophthalmic lens such as spectacle glasses, contacts, or IOLs. FIG. 1 shows lens 20, being an augmented monofocal lens of the present disclosure, as an IOL comprising optic 22 and haptics 24 and further comprising paraxial region 26 about the optical axis and monofocal region 28 from the extent of paraxial region 26 to the edge of optic 22. The radius of paraxial region 26, R₀, is shown as a dashed line in FIG. 1. Within paraxial region 26, a wavefront perturbation has been added. The wavefront perturbation could be realized with surface relieving on either the anterior and posterior surfaces of optic 22 or with refractive index modification of the material within optic 22. The objective of the wavefront perturbation is to extend the depth of focus that may correct the presbyopia of the wearer under photopic conditions at all distances from far to near continuously. The perturbation of the wavefront at either the anterior and posterior surfaces of the optic or with refractive index modification of the lens material at some depth inside the optic is a continuous even function of the radius of the aperture of the wavefront. Therefore, it is also rotationally symmetrical about the optical axis and is continuously differentiable within paraxial region 26. The wavefront of the augmented monofocal lens W(r) consists of two parts, the one is the base monofocal wavefront, Wₐ(r); the other is Wₚ(r), the perturbation wavefront. This is expressed in Eq. (1).

W(r)=Wₐ(r)+Wₚ(r) Eq. (1)

The base monofocal wavefront Wₐ(r) is normally a standard aspheric wavefront as expressed in Eq. (2)

$${W}_{a} ( r ) = \frac{c {r}^{2}}{1 + \sqrt{1 - ( 1 + k ) {c}^{2} {r}^{2}}} + {\sum }_{i = 1}^{i = n} {a}_{i} {r}^{2 i} \qquad Eq . ( 2 )$$

The perturbation part of the wavefront is confined within the paraxial region 26 that has a radius of R₀. Beyond the paraxial region 26 from R₀ to the edge of the lens aperture of radius R, the probation is a constant C, as is shown in Eq. (3).

Furthermore, the first-order derivative of the perturbed wavefront is also:

$${W}_{p} ( r ) = \{ \begin{matrix}f ( r ) & 0 \leq r < {R}_{0} \\ C & {R}_{0} \leq r \leq R\end{matrix} \qquad Eq . ( 3 )$$

where
R is the radius of the lens aperture;
R₀ is the radius of the aperture of the perturbation as is shown in FIG. 1;
r is the distance from the optical axis; and
ƒ(r) is a continuous even function of r.

In the engineering design of the wavefront perturbation for the desired performances, ƒ(r) must be parameterized. The following are at least three ways to parameterize ƒ(r). ƒ(r) may be parameterized using a cosine series as indicated in Eq. (4) below:

$$f ( r ) = {\sum }_{n = 0}^{N} {a}_{n} cos ( n ω r ) \qquad Eq . ( 4 )$$

where:

ω=2π/D Eq. (5)
r is the distance from the optical axis; and
D is the diameter of paraxial region 26, or simply 2R₀.

In the design of the wavefront perturbation, N, the order of the cosine series, together with an where (n=0, 1, 2, . . . , N), are to be optimized for the wavefront perturbation ƒ(r) to achieve the desired performance of lens 20. Optimal wavefront perturbations expressed as a cosine series would have N in the range of 3≤N≤10, R₀ in the range of 0.5 mm≤R₀ 2 mm, and aₙ the range of −0.5≤aₙ≤0.5. The following paragraphs discloses two embodiments of the wavefront perturbation expressed as a cosine series.

The first embodiment of the wavefront perturbation, expressed as a cosine series, has the parameters shown in Table 1.

TABLE 1
Parameters for the second embodiment of the wavefront perturbation,
expressed as a cosine series. R₀ is measured in millimeters,
the coefficients of the cosine series are measured in waves.

| Parameter | Value |
|---|---|
| R₀ | 1.130697 |
| a₀ | 0 |
| a₁ | −0.19033 |
| a₂ | 0.012961 |
| a₃ | −0.12154 |
| a₄ | 0.166943 |
| a₅ | −0.03089 |
| a₆ | 0.054495 |
| a₇ | −0.04147 |
| a₈ | 0.102731 |

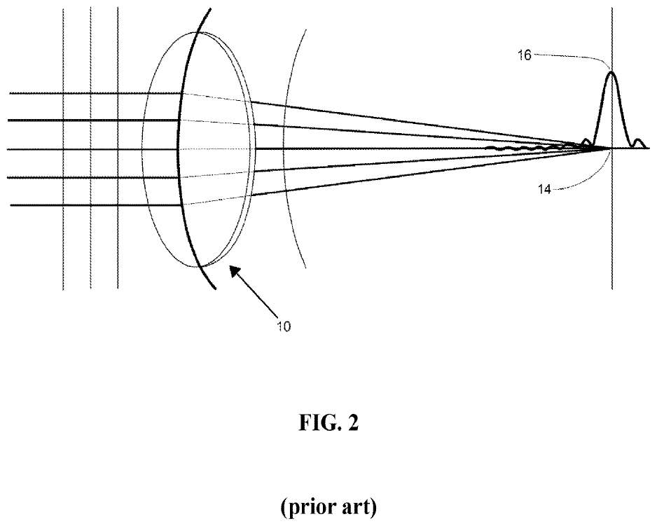

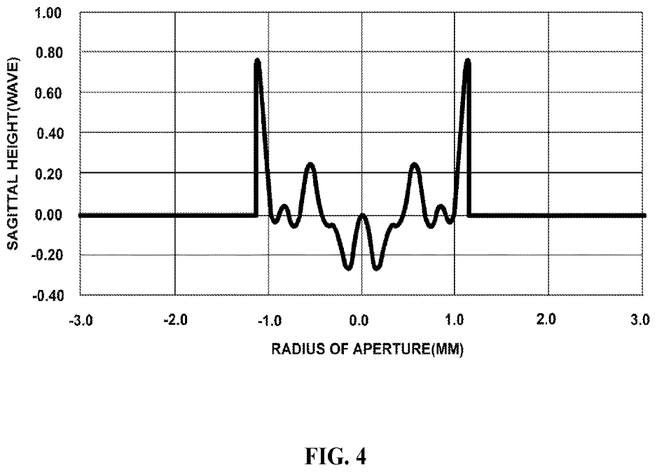

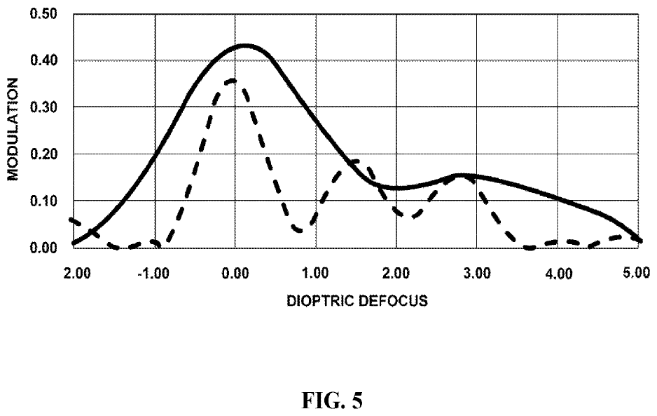

FIG. 4 shows the resulting wavefront perturbation sag for the first embodiment on the surface of optic 22, with the x-axis being the surface of optic 22 and the y-axis being the height of the sag. The optical axis of optic 22 is at 0.0 mm on the x-axis. The perturbed wavefront sag is horizontal outside of R₀, where R₀ is the radius of paraxial region 26, as shown in FIG. 1. In FIG. 5, the through focus MTF is shown for the perturbed wavefront of the first embodiment at a 3 mm aperture, or pupil size of the eye. The solid line shows the through focus MTF at 25 cycles per millimeter (CPMM), while the dashed line shows the through focus MTF at 50 CPMM. Comparing the through focus MTF from FIG. 5 with monofocal through focus MTF 16 in FIG. 2, it may be seen that the wavefront perturbation of the first embodiment has an expanded depth of focus as compared to the prior art monofocal lens shown in FIG. 2.

The second embodiment of the wavefront perturbation, also expressed as a cosine series, has the parameters shown in Table 2.

TABLE 2
Parameters for the second embodiment of the wavefront perturbation,
expressed as a cosine series. R₀ is measured in millimeters,
the coefficients of the cosine series are measured in waves.

| Parameter | Value |
|---|---|
| R₀ | 1.25 |
| a₀ | −0.41004 |
| a₁ | −0.17767 |
| a₂ | −0.11494 |
| a₃ | −0.0313 |
| a₄ | −0.01677 |
| a₅ | 0.095931 |
| a₆ | −0.10124 |
| a₇ | 0.067486 |
| a₈ | −0.00509 |

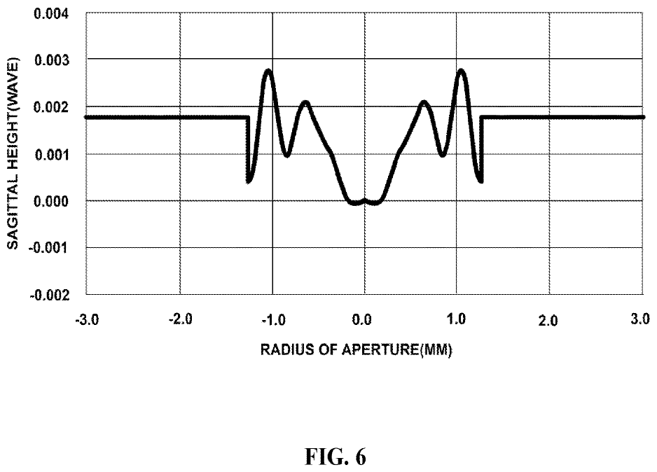

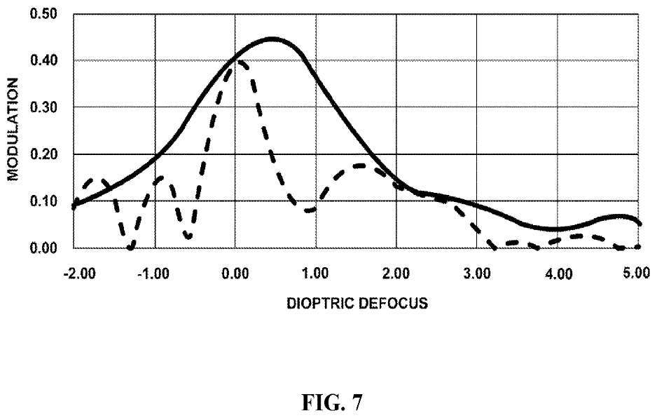

FIG. 6 shows the resulting wavefront perturbation sag for the second embodiment on the surface of optic 22, with the x-axis being the surface of optic 22 and the y-axis being the height of the sag. The optical axis of optic 22 is at 0 0 mm on the x-axis. The perturbed wavefront sag is horizontal outside of R₀, where R₀ is the radius of paraxial region 26, as shown in FIG. 1. In FIG. 7, the through focus MTF is shown for the perturbed wavefront of the second embodiment at a 3 mm aperture. The solid line shows the through focus MTF at 25 cycles per millimeter (CPMM), while the dashed line shows the through focus MTF at 50 CPMM. Comparing the through focus MTF from FIG. 7 with monofocal through focus MTF 16 in FIG. 2, it may be seen that the wavefront perturbation of the second embodiment has an expanded depth of focus as compared to the prior art monofocal lens shown in FIG. 2.

The wavefront perturbation function ƒ(r) may also be expressed as a Fourier harmonics series as indicated in Eq. (6).

$$f ( r ) = {\sum }_{i = 1}^{i = M} {α}_{i} {r}^{2 i} + {\sum }_{n = 1}^{N} ( {a}_{n} cos ( n ω {r}^{2} ) + {b}_{n} sin ( n ω {r}^{2} ) ) \qquad Eq . ( 6 )$$

Equivalently, Eq. (6) can be expressed in the form of Eq. (7)

$$f ( r ) = {\sum }_{i = 1}^{i = M} {α}_{i} {r}^{2 i} + {\sum }_{n = 1}^{N} {A}_{n} cos ( n ω {r}^{2} + {ϕ}_{n} ) \qquad Eq . ( 7 )$$

where:

ω=πP/λ Eq. (8)
M is an integer indicating the order of the first polynomial.
N is an integer indicating the order of the Fourier harmonic series;
λ is the wavelength of incident light; and
r is the distance from the optical axis.

In the engineering design of the wavefront perturbation, M, αᵢ (i=1, 2, . . . M), and N, together with P, Aₙ, φₙ (n=0, 1, 2, . . . , N) are to be decided for the wavefront perturbation ƒ(r) to achieve the desired lens performances.

Just as with the cosine wavefront perturbation, there exists a range of values for each design parameter of the Fourier series wavefront perturbation. Normally, R₀ is in the range of 0.5 mm≤R₀≤2.5 mm; M is in the range of 1≤M≤3, N is in the range of 5≤N≤15, Aₙ is in the range of −0.5≤Aₙ≤0.5, and φₙ is in the range of −π≤φₙ≤π. Though much higher orders of both M and N could be used but with minimal values of the corresponding coefficients.

The third embodiment of the wavefront perturbation, expressed as a Fourier harmonic series, has the following parameters: P=0.56 (m⁻¹), R₀=1.083 (mm) while Table 3 contains the Fourier harmonic parameters. The asphere parameters in this embodiment is α₁=−1.02564.

TABLE 3
The Fourier harmonic parameters for the third
embodiment of the wavefront perturbation.

| n | Aₙ(wave) | φₙ |
|---|---|---|
| 1 | 0.05769920 | 0.24211907 |
| 2 | 0.23426701 | −1.66149706 |
| 3 | 0.03800426 | −1.45079958 |
| 4 | −0.02373634 | 1.55496512 |
| 5 | 0.02734461 | −0.57577025 |
| 6 | 0.03739066 | −1.51366902 |
| 7 | −0.01797941 | 1.55055861 |
| 8 | −0.01337226 | 1.60851219 |
| 9 | 0.00841704 | −1.02274817 |
| 10 | −0.01114590 | 1.62044934 |
| 11 | −0.00712054 | 1.04770549 |
| 12 | 0.00978682 | 0.00000000 |

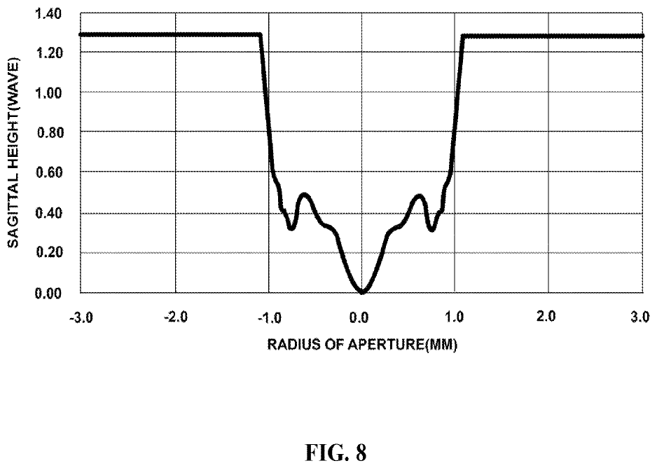

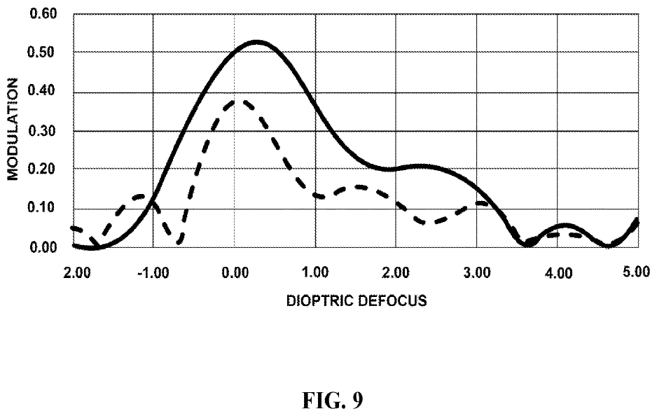

FIG. 8 shows the resulting wavefront perturbation sag for the third embodiment on the surface of optic 22, with the x-axis being the surface of optic 22 and the y-axis being the height of the sag. The optical axis of optic 22 is at 0 0 mm on the x-axis. The perturbed wavefront sag is horizontal outside of R₀, where R₀ is the radius of paraxial region 26, as shown in FIG. 1. In FIG. 9, the through focus MTF is shown for the perturbed wavefront of the third embodiment at a 3 mm aperture. The solid line shows the through focus MTF at 25 cycles per millimeter (CPMM), while the dashed line shows the through focus MTF at 50 CPMM. Comparing the through focus MTF from FIG. 9 with monofocal through focus MTF 16 in FIG. 2, it may be seen that the wavefront perturbation of the third embodiment has an expanded depth of focus as compared to the prior art monofocal lens shown in FIG. 2.

The fourth embodiment of the wavefront perturbation, expressed as a Fourier harmonic series, has the following parameters: P=0.5 (m⁻¹), λ=0.546 (μm), R₀=1.32 (mm) while Table 4 contains the Fourier harmonic parameters. The asphere parameters in this embodiment is α₁=−1.37363.

TABLE 4
The Fourier harmonic parameters for the fourth
embodiment of the wavefront perturbation.

| n | Aₙ(wave) | φₙ |
|---|---|---|
| 1 | −0.23211663 | 0.17187473 |
| 2 | −0.11834860 | 0.43796103 |
| 3 | −0.15291156 | 3.13271012 |
| 4 | 0.11131130 | −1.57941008 |
| 5 | −0.03540262 | 0.61866139 |
| 6 | −0.03266607 | −0.54495068 |
| 7 | 0.05101538 | 0.18622125 |
| 8 | 0.04045728 | −0.60008160 |
| 9 | −0.02798241 | 1.42388795 |
| 10 | −0.02751885 | −0.51448718 |
| 11 | 0.01893362 | 0.57888249 |
| 12 | 0.02482207 | −0.37403284 |

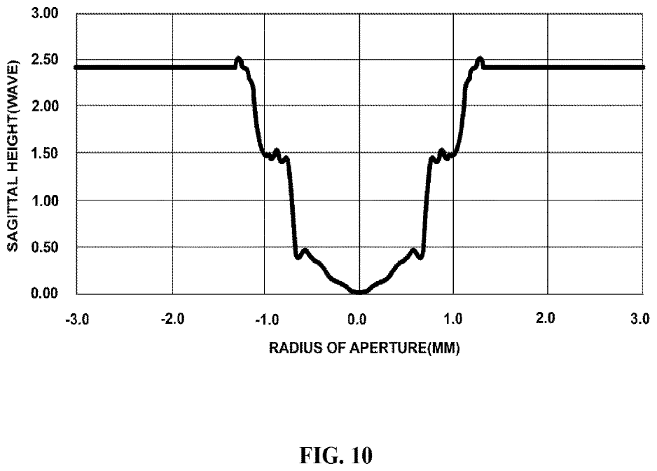

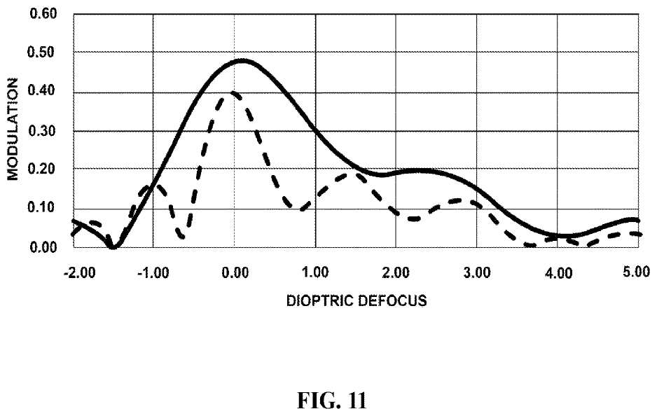

FIG. 10 shows the resulting wavefront perturbation sag for the fourth embodiment on the surface of optic 22, with the x-axis being the surface of optic 22 and the y-axis being the height of the sag. The optical axis of optic 22 is at 0.0 mm on the x-axis. The perturbed wavefront sag is horizontal outside of R₀, where R₀ is the radius of paraxial region 26, as shown in FIG. 1. In FIG. 11, the through focus MTF is shown for the perturbed wavefront of the fourth embodiment at a 3 mm aperture. The solid line shows the through focus MTF at 25 cycles per millimeter (CPMM), while the dashed line shows the through focus MTF at 50 CPMM. Comparing the through focus MTF from FIG. 11 with monofocal through focus MTF 16 in FIG. 2, it may be seen that the wavefront perturbation of the fourth embodiment has an expanded depth of focus as compared to the prior art monofocal lens shown in FIG. 2.

The fifth embodiment of the wavefront perturbation, expressed as a Fourier harmonic series, has the following parameters P=0.5 (m⁻¹), λ=0.546 (μm), R₀=1.45 (mm) while Table 5 contains the Fourier harmonic parameters. The asphere parameters in this embodiment is α₁=−1.37363.

TABLE 5
The Fourier harmonic parameters for the fifth
embodiment of the wavefront perturbation.

| n | Aₙ(wave) | φₙ |
|---|---|---|
| 1 | 0.09586816 | −0.59204769 |
| 2 | 0.17105876 | −0.32425476 |
| 3 | 0.18663822 | −2.10625634 |
| 4 | 0.01392298 | −0.09599993 |
| 5 | 0.04626154 | −0.31317129 |
| 6 | −0.00163297 | 2.16857195 |
| 7 | 0.03873800 | −0.01407355 |
| 8 | 0.04001933 | −1.08303727 |
| 9 | −0.02100139 | −1.06656949 |
| 10 | −0.01767998 | −2.95188393 |
| 11 | −0.00923541 | 1.79818159 |
| 12 | 0.01482980 | 0.71341635 |

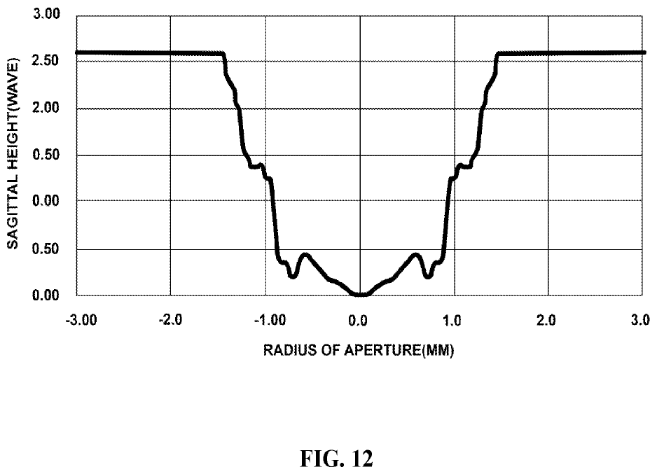

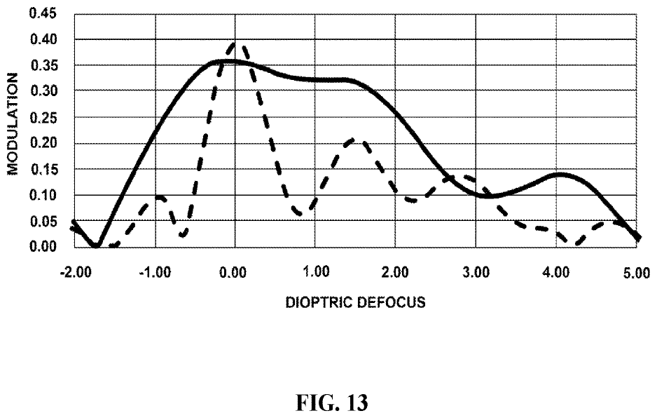

FIG. 12 shows the resulting wavefront perturbation sag for the fifth embodiment on the surface of optic 22, with the x-axis being the surface of optic 22 and the y-axis being the height of the sag. The optical axis of optic 22 is at 0.0 mm on the x-axis. The perturbed wavefront sag is horizontal outside of R₀, where R₀ is the radius of paraxial region 26, as shown in FIG. 1. In FIG. 13, the through focus MTF is shown for the perturbed wavefront of the fifth embodiment at a 3 mm aperture. The solid line shows the through focus MTF at 25 cycles per millimeter (CPMM), while the dashed line shows the through focus MTF at 50 CPMM. Comparing the through focus MTF from FIG. 13 with monofocal through focus MTF 16 in FIG. 2, it may be seen that the wavefront perturbation of the fifth embodiment has an expanded depth of focus as compared to the prior art monofocal lens shown in FIG. 2.

The wavefront perturbation function ƒ(r) may also be expressed as an even Taylor series as indicated in Eq. (9). It is infinitely differentiable at the center of the lens aperture with r=0.

$$f ( r ) = {\sum }_{n = 0}^{N} {p}_{n} {r}^{2 n} \qquad Eq . ( 9 )$$

where:
pₙ is the coefficient of the Taylor series; and
r is the distance from the optical axis of the optics.

In the engineering design of the wavefront perturbation, N, the order of the Taylor series, together with pₙ (n=0, 2, 3, . . . , N), are to be optimized for the wavefront perturbation to achieve the desired performance of the ophthalmic lens.

The wavefront perturbation function ƒ(r) may also be expressed as even Zernike polynomials as indicated in Eq. (10).

$$f ( r ) = {\sum }_{n = 0}^{N} {z}_{n}^{m} {Z}_{n}^{m} ( r , θ ) \qquad Eq . ( 10 )$$

where:
r and θ are the radial and azimuth polar coordinates on the lens aperture;
r is the radius of the lens aperture;
n and m are integer variables indicating the radial and azimuth order of the polynomials;

$${Z}_{n}^{m}$$

are the corresponding coefficients; and

$${Z}_{n}^{m} ( r , θ )$$

are the so-called Zernike polynomials.

Since ƒ(r) is rotationally symmetric, the coefficients of the non-zero azimuth orders in Eq. (10) must be all zero. In other words, the Zernike polynomials shall only consist of the m=0 terms.

$${{{Z}_{n}^{m} ( r , θ ) \vert}_{m = 0} = {R}_{n}^{m} ( r ) cos ( m θ ) \vert}_{m = 0} = {R}_{n}^{0} ( r ) \qquad Eq . ( 11 )$$

$${R}_{n}^{0} ( r ) = {\sum }_{k = 1}^{n / 2} \frac{{( - 1 )}^{k} ( n - k ) !}{k ! ( \frac{n}{2} - k ) ! ( \frac{n}{2} - k ) !} {r}^{n - 2 k} \qquad Eq . ( 12 )$$

Since ƒ(r) is rotationally symmetric, the coefficients of the non-zero azimuth orders in Eq. (11) must be all zero. In other words, the Zernike polynomials shall only consist of the m=0 terms. The desired Zernike polynomial form of the wavefront perturbation can then be expressed with Eq. (13).

$$f ( r ) = {\sum }_{n = 0}^{N} {z}_{n} ( {\sum }_{k = 1}^{n / 2} \frac{{( - 1 )}^{k} ( n - k ) !}{k ! ( \frac{n}{2} - k ) ! ( \frac{n}{2} - k ) !} {r}^{n - 2 k} ) \qquad Eq . ( 13 )$$

The following are the explicit expressions of the first 20 terms of the rotationally symmetric Zernike polynomials.

$${Z}_{0}^{0} ( r ) 1 \qquad Eq . ( 14 )$$

$${Z}_{2}^{0} ( r ) 2 {r}^{2} - 1 \qquad Eq . ( 15 )$$

$${Z}_{4}^{0} ( r ) 6 ( {r}^{4} - {r}^{2} ) + 1 \qquad Eq . ( 16 )$$

$${Z}_{6}^{0} ( r ) 20 {r}^{6} - 30 {r}^{4} + 12 {r}^{2} - 1 \qquad Eq . ( 17 )$$

$${Z}_{8}^{0} ( r ) 70 {r}^{8} - 140 {r}^{6} + 90 {r}^{4} - 20 {r}^{2} + 1 \qquad Eq . ( 18 )$$

$${Z}_{10}^{0} ( r ) 252 {r}^{10} - 630 {r}^{8} + 560 {r}^{6} - 210 {r}^{4} + 30 {r}^{2} - 1 \qquad Eq . ( 19 )$$

$${Z}_{12}^{0} ( r ) 924 {r}^{12} - 2772 {r}^{10} + 3150 {r}^{8} - 1680 {r}^{6} + 420 {r}^{4} - 42 {r}^{2} + 1 \qquad Eq . ( 20 )$$

$${Z}_{14}^{0} ( r ) 3432 {r}^{14} - 12012 {r}^{12} + 16632 {r}^{10} - 11550 {r}^{8} + 4200 {r}^{6} - 756 {r}^{4} + 56 {r}^{2} - 1 \qquad Eq . ( 21 )$$

$${Z}_{16}^{0} ( r ) 12870 {r}^{16} - 51480 {r}^{14} + 84084 {r}^{12} - 72072 {r}^{10} + 34650 {r}^{8} - 9240 {r}^{6} + 1260 {r}^{4} - 72 {r}^{2} + 1 \qquad Eq . ( 22 )$$

$${Z}_{18}^{0} ( r ) 4862 \; 0 {r}^{18} - 218790 {r}^{16} + 411840 {r}^{14} - 420420 {r}^{12} + 252252 {r}^{10} - 90090 {r}^{8} + 18480 {r}^{6} - 1980 {r}^{4} + 90 {r}^{2} - 1 \qquad Eq . ( 23 )$$

$${Z}_{20}^{0} ( r ) 184756 {r}^{20} - 923780 {r}^{18} + 1969110 {r}^{16} - 2333760 {r}^{14} + 1681680 {r}^{12} - 756756 {r}^{10} + 210210 {r}^{8} - 34320 {r}^{6} + 2970 {r}^{4} - 110 {r}^{2} + 1 \qquad Eq . ( 24 )$$

Zernike polynomial forms of higher orders can be generated with Eq. (12) and there are already provided in industry optical design software such as the Zemax OpticStudio® software.

In the engineering design of the wavefront perturbation, N, the order of the Zernike polynomials, together with zₙ (n=0, 1, 2, . . . , N) are to be optimized for the wavefront perturbation to achieve the desired performance of the ophthalmic lens.

The rotational Zernike polynomial wavefront perturbation in Eq. (13) is effectively the same as the Taylor series in Eq. (9). However, the wavefront perturbation expressed in Eq. (13) may make the engineering design converge faster to the optimal wavefront perturbation for the desired IOL performance.

Based on the mathematical principles of the Taylor series, both the cosine series and Fourier harmonic series types of wavefront perturbations can also be expressed in the form of Taylor series, and hence, Zernike polynomials. Nevertheless, the order of the equivalent Taylor Series will be much higher than the order of the cosine and Fourier harmonic series.

Exemplary embodiments of the invention have been disclosed in an illustrative style. Accordingly, the terminology employed throughout should be read in a non-limiting manner. Although minor modifications to the teachings herein will occur to those well versed in the art, it shall be understood that what is intended to be circumscribed within the scope of the patent warranted hereon are all such embodiments that reasonably fall within the scope of the advancement to the art hereby contributed and that that scope shall not be restricted, except in the light of the appended claims and their equivalents.

What is claimed is:

1. An ophthalmic lens, comprising
an optic comprising
an anterior surface,
a posterior surface,
an edge at its periphery,
and an optical axis,
where at least one of the anterior surface and the posterior surface comprising:
a first zone extending from the optical axis to a radial boundary having an aspheric wavefront and a perturbation wavefront, the perturbation wavefront expressed as a continuous even function and
a second zone extending from the radial boundary to the edge having an aspheric wavefront.

2. The ophthalmic lens of claim 1 wherein the perturbation wavefront is rotationally symmetrical about the optical axis.

3. The ophthalmic lens of claim 1 wherein the perturbation wavefront is realized with surface relieving on either the anterior and posterior surfaces of the optic.

4. The ophthalmic lens of claim 1 wherein the perturbation wavefront is realized with refractive index modification of the material within the optic.

5. The ophthalmic lens of claim 1 wherein the continuous even function of the perturbation wavefront is expressed as a cosine series of the form:
$$f ( r ) = {\sum }_{n = 0}^{N} {a}_{n} cos ( n ω r )$$
where:
ω=2π/D
r is the distance from the optical axis; and
D is the diameter of the first zone.

6. The ophthalmic lens of claim 5 wherein the cosine series would have N in the range of 3≤N≤10, R₀ in the range of 0.5 mm≤R₀≤2 mm, and aₙ the range of −0.5≤aₙ≤0.5.

7. The ophthalmic lens of claim 1 wherein the continuous even function of the perturbation wavefront is expressed as a Fourier harmonics series of the form:
$$f ( r ) = {\sum }_{i = 1}^{i = M} {α}_{i} {r}^{2 i} + {\sum }_{n = 1}^{N} {A}_{n} cos ( n ω {r}^{2} + {ϕ}_{n} )$$
where:
ω=πP/λ
M is an integer indicating the order of the first polynomial.
N is an integer indicating the order of the Fourier harmonic series;
λ is the wavelength of incident light; and
r is the distance from the optical axis.

8. The ophthalmic lens of claim 7 wherein the Fourier harmonics series would have R₀ in the range of 0.5 mm≤R₀≤2.5 mm; M in the range of 1≤M≤3, N in the range of 5≤N≤15, Aₙ in the range of −0.5≤Aₙ≤0.5, and φₙ in the range of −π≤φₙ≤π.

9. The ophthalmic lens of claim 1 wherein the continuous even function of the perturbation wavefront is expressed as an even Taylor series of the form:
$$f ( r ) = {\sum }_{n = 0}^{N} {p}_{n} {r}^{2 n}$$
where:
pₙ is the coefficient of the Taylor series; and
r is the distance from the optical axis of the optics.

10. The ophthalmic lens of claim 1 wherein the continuous even function of the perturbation wavefront is expressed as an even Zernike polynomials of the form:
$$f ( r ) = {\sum }_{n = 0}^{N} {z}_{n}^{m} {Z}_{n}^{m} ( r , θ )$$
where:
r and θ are the radial and azimuth polar coordinates on the lens aperture;
r is the radius of the lens aperture;
n and m are integer variables indicating the radial and azimuth order of the polynomials;
$${z}_{n}^{m}$$
are the corresponding coefficients; and
$${Z}_{n}^{m} ( r , θ )$$
are the so-called Zernike polynomials.
